(19) United States
(12) Patent Application Publication
(10) Pub. No.: US 2018/0070972 A1
(43) Pub. Date: Mar. 15, 2018

(54) Instrument For Use In Laparoscopy

(71) Applicants: Stan Chao, Albuquerque, NM (US); Zefr Chao, Albuquerque, NM (US)

(72) Inventors: Stan Chao, Albuquerque, NM (US); Zefr Chao, Albuquerque, NM (US)

(21) Appl. No.: 15/698,781

(22) Filed: Sep. 8, 2017

Related U.S. Application Data
(60) Provisional application No. 62/393,343, filed on Sep. 12, 2016.

(30) Foreign Application Priority Data
Jul. 25, 2017 (GB) GB1711981.9

(51) Int. Cl. A61B 17/3205

(52) U.S. Cl. CPC A61B 17/32053; A61B 17/320016

(57) ABSTRACT

An instrument 10 for use in laparoscopic surgery comprises a circular cutting arrangement 12 for making a circular cut to fascia; wherein the circular cutting arrangement 12 is circularly rotatable, to facilitate cutting through fascia.

### REFERENCES TO RELATED APPLICATIONS

This application claims priority to application Ser. No. 62/393,343 filed on Sep. 12, 2016 entitled CHAOSTAT and also claims priority of British Patent Application |GB| number 1711981.9 filed on Jul. 25, 2017 entitled INSTRUMENT FOR USE IN LAPAROSCOPY the disclosures of which are incorporated herein by reference.

### TERMS/DEFINITIONS

‘Arrangement’:

The term arrangement is a technical term which means a feature, or combination of features, (usually with a given purpose). For example, a ‘measuring arrangement’ is a feature or features, to facilitate measuring. A ‘holding arrangement’ is a feature or features, to facilitate holding.

‘Feature’:

The term ‘feature’ is a broad term that includes within its scope any feature under the Sun. A feature may, for example, be an element, body, member, or may even be an aperture (ie opening/hole or the such like). Thus the term ‘holding feature’ includes within its scope, for example, any element, body, member, or even aperture, to facilitate holding. A ‘feature’ may also be a ‘part’ of a broader feature; for example, if a part of an invention is disclosed/claimed as comprising a ‘padding feature’, this does not limit the padding feature to being a separate feature that is added to the invention (eg a sponge element, adhered to the invention); the invention, for example, may have a part made of sponge, or have an inner sponge (or soft) layer—in such a case, if that part of the invention is clearly of a material/nature that provides padding, then this falls within a scope of what is defined in the present application as a ‘padding feature’.

The or Each

The term ‘the or each’ (either in disclosure and/or a claim) can refer back to a single feature/thing, and/or can refer back to a plurality of features/things. When the term is read as referring back to a plurality of features/things, it should be taken as meaning, and including within its scope, ‘at least one, or more, or all (ie each)’ of the said features/things. Thus, to give an example, if a square is referred to/disclosed that has four corners, if the term ‘the or each corner’ is used, it includes within its scope ‘one of the corners, (or two, or three, or all of the corners)’.

When any one feature/thing is afforded any feature(s)/definition in the present application, it is taken as read that, where a plurality of the said feature/thing is provided, ‘the or each’ said feature/thing may be provided/claimed comprising the said feature(s)/definition (ie at least one, or more or all). Similarly, wherever a plurality of the said feature/thing are afforded any feature(s)/definition, it is taken as read that ‘the or each’ said feature/thing may be provided/claimed comprising the feature(s)/definition (ie at least one, or more, or all of the plurality), and/or that even just one said feature/thing may be provided/claimed comprising the feature(s)/definition in an embodiment/claim wherein an invention is defined as comprising ‘a’ (eg singular) said feature/thing.

Reference to Multiple Similar Elements in Plural

In the present application, there may be provided/numbered features wherein the same primary number is used, with a suffix. For example, a first side of the/an invention may be numbered 600′ (for example), and a second side of the invention numbered 600″ (for example). In any such case (or where any other suffix is used, such as I′ and ‘R’ to denote ‘left’ and ‘right’) when such features are referred to together (ie ‘the sides’), the plural may be numbered/referred to with the primary number (without the suffix). Thus the first side 600′ and second side 600″ may, for example, be referred to simply as ‘the sides 600’. Furthermore, if a side is referred to (not specifying which one of the sides), the primary number (ie 600, for example) may be used, without any suffix.

### BACKGROUND (PROBLEMS)

### Field

The present invention relates to an instrument for use in laparascopy. How to cut through fascia, without damaging internal structures and/or organs of a patient, is a subject that is explored.

Laparoscopy is a type of surgery which is intended to minimise invasiveness of surgery, making a relatively small incision to a patient, and typically entering light, camera, and various surgical instrument(s) through the incision, to carry out surgery. Such are the benefits of laparoscopy that approximately 7.5 million (or more) laparoscopic surgeries are carried out per year, worldwide.

However, there is a problem with laparoscopic surgery. A surgeon needs to penetrate various tissues, (including fascia), to gain access, internally, to the body. Typically, surgery begins with making a skin incision to a patient. (This is typically done by using a scalpel). The surgeon must then penetrate through various anatomical layers in order to gain entry into the patient. For example, in abdominal laparoscopic surgery, the surgeon must penetrate through various anatomical layers of the abdominal wall, into the peritoneal cavity. The most notable resistance (eg to intra-abdominal entry, in the case of abdominal laparoscopic surgery, but also in other laparoscopic surgeries) is a tough layer of tissue called the ‘fascia’. Tissues below the fascia may include muscle tissue(s), parietal peritoneum, and may include extra peritoneal fat.

However, the fascia can be tough. Therefore, typically, a surgeon uses a pointed instrument, (such as a trocar), which tends to have a relatively sharp (pointed) tapered tip, to penetrate through the fascia. The problem is, even with a tapered instrument such as this, often significant inward force is required in order to penetrate, and ‘puncture through’, the fascia.

Because such force is required in order to puncture through the fascia, and because such instruments tend to be sharp/pointed, a significant problem can occur whereby the surgeon, in using such downward/inward force, not only penetrates the fascia, but fails to stop the downward/inward pressure once the fascia is penetrated, with the tapered/pointed tip of the instrument carrying through further, and either damaging internal structures of the patient, or, worse still, puncturing internal structures inside the body of the patient.

In layman's terms, there is a problem whereby the pointed instrument may ‘burst through’ the fascia, and carry on in the body, then penetrating or damaging internal structures and/or organs of the body.

The problem is exacerbated by the points/tips of such instruments becoming blunted, which can lead to even more force being required to penetrate through the fascia. This leads to even more likelihood of the surgeon penetrating (and bursting) through the fascia, and going on to do internal damage to internal structures inside the body.

Use of instruments that have a singular sharply tapered tip, (where load of the pressure is therefore focused into a very small (and sharp) cutting surface), seems to be part of the problem.

It is well known that approximately 50% of all laparoscopic complications occur in the initial penetration through the fascia. To give an example of the amount of injuries (in some case, serious injuries/complications) caused to patients, due to the above problem(s), taking the example of abdominal laparoscopic surgery alone, 1.8 major injuries to the bowel are caused per 1000 abdominal laparoscopies. These injuries include severe injuries such as penetration of the bowel. Whilst 1.8 bowel injuries per 1000 laparoscopies may seem like a small number, this amounts to 1800 such bowel injuries per one million laparoscopies. Given that laparoscopy is widespread, with approximately 7.5 million total laparoscopies being carried out per year, even these small percentages amount to a large amount of patient injuries. There are also approximately 1000 vascular injuries caused by the aforementioned problem(s), per million laparoscopies.

Thus it would be desirable if instrument(s) could be provided that reduce (or eliminate) risk of injury to internal structures of the patient's body.

### SUMMARY

The present invention is defined by the accompanying claims, to which reference should now be made.

Examples of the present invention seek to provide a solution to any or all of the above problem(s) by providing: an instrument for use in laparoscopic surgery, comprising: a circular cutting arrangement for making a circular cut to fascia; wherein the circular cutting arrangement is circularly rotatable, to facilitate cutting through fascia.

The circular cutting arrangement is rotatable, to cut through fascia. Therefore load can be spread around the circular cutting arrangement. This is in contrast to sharp singular pointed/tapered instruments (as previously discussed) which are used to penetrate through the fascia.

Furthermore, because the instrument uses circular motion to effect cutting, (and not only downward, or ‘inward’, force), when the circular cutting arrangement is not being rotated, it can be relatively harmless. For example, with previous instruments that have a tapered singular point to penetrate through fascia, if one were to press such a point against one's arm, for example, it would quickly become painful, and could penetrate through skin. However, if the present circular cutting arrangement is pressed against one's arm, it is feasible that the cutting arrangement, because load is spread over the circular cutting arrangement and/or because it is not being rotated, will not cause discomfort and/or penetrate through skin, (unless significant force were applied). Thus one other potential benefit of the instrument is that, by relying, to some extent, on circular motion, rather than simply downward (or ‘inward’) force, it makes it less likely that the surgeon will ‘burst through’ the fascia, and engage internal structures and/or organs unintentionally. Furthermore, (for reasons already stated), even if such an event occurred, (ie that internal structures and/or organs were unintentionally engaged with the circular cutting arrangement), it is far less likely that it will cause any damage.

Furthermore, a circular cutting arrangement and motion is more controllable than an incision made by a sharp/tapered single point. This again makes it less likely a surgeon, using such a circular cutting arrangement, will ‘burst through’ the fascia, causing internal injury.

It is thought the instrument will lessen (and may eliminate, wholly) any injuries caused in laparoscopic surgery by the aforementioned problem(s).

Preferably the instrument is able to spread a cut made by the cutting arrangement. Thus, preferably, the instrument comprises a cut-spreading arrangement, for spreading a cut made by the circular cutting arrangement. (This may be particularly useful for spreading fascia).

According to a second aspect of the invention, (referring to a method), examples of the second aspect seek to provide a solution by providing: a method for carrying out laparoscopic surgery, comprising: introducing a circular cutting arrangement to a cut point on a patient; and circularly rotating the circular cutting arrangement, to make a circular cut through fascia.

The method may draw upon any disclosure and/or feature(s) of the instrument in the present application, and/or may draw upon any disclosure of the present application. (The or a method may be claimed as a method for cutting fascia. The or a method may be claimed as a method for cutting fascia for laparoscopy).

The second aspect (or any further aspect(s)) may comprise any of the feature(s) of the first aspect and may draw upon any of the feature(s) and/or disclosure of the present application, as optional and/or preferable feature(s). Any aspect may comprise any feature(s) of any other aspect(s), whether the feature(s) be essential or preferable and/or optional to the other aspect(s).

### BRIEF DESCRIPTION OF THE DRAWINGS

Examples of the present invention will now be more particularly described, with reference to the accompanying drawings, by way of example only, in no way limiting a scope of the invention, in which:

FIG. 1 is a perspective angled view, from below, of an example of an instrument for use in laparoscopic surgery, comprising: a circular cutting arrangement for making a circular cut to fascia; wherein the circular cutting arrangement is circularly rotatable, to facilitate cutting through fascia;

FIG. 2 is a side view of a same example embodiment as FIG. 1;

FIG. 3 is a close-up view, from below, focusing solely on an example of the circular cutting arrangement, showing an embodiment wherein the circular cutting arrangement comprises two cut parts, shown in a closed position;

FIG. 4 is a close-up view, from below, of a same example embodiment as shown in FIG. 3, now showing the circular cutting arrangement (and cut parts) in an open position;

FIG. 5 is a close-up view, from below, of an example embodiment of the circular cutting arrangement, wherein the circular cutting arrangement comprises four cut parts;

FIG. 6 is a reverse side view of a same or similar example embodiment as shown in FIG. 1 and FIG. 2, now showing the cutting arrangement opened, and spreading a cut made to fascia, and also showing a cross-sectional representational view of tissues of a patient;

FIG. 7 is a side profile view of a same/similar embodiment to what is shown in FIG. 1, FIG. 2, and FIG. 6, showing an example of the instrument being curved; and

FIG. 8 is a basic flowchart, showing an example embodiment of a method for performing laparoscopic surgery.

Note: At some patent offices, when claim(s) are allowed for patent, it is required that Figures that do not show all the feature(s) of the/an invention claimed (and that are not within a scope of what is claimed) are denoted as ‘not being claimed’ (or words to that effect). Even in such case, it will be obvious that such Figure(s), even if denoted as ‘not being claimed’ (or words to that effect) in the Brief Description of The Drawings, may or do show some or many preferable and/or optional feature(s) of the/an invention claimed. Thus such Figure(s) (and/or disclosure related to such Figure(s) and/or embodiment(s)), should nevertheless be considered relevant (and/or may be relevant) to the/an invention claimed.

### DETAILED DESCRIPTION

Referring to the drawings, there is shown an instrument 10 for use in laparoscopic surgery, comprising: a circular cutting arrangement 12 for making a circular cut to fascia; wherein the circular cutting arrangement 12 is circularly rotatable, to facilitate cutting through fascia.

Preferably a portion or a whole of the instrument 10 is circularly rotatable to circularly rotate the circular cutting arrangement 12. This is seen in the example embodiments. In the example embodiments shown, the whole of the instrument 10 is circularly rotatable to circularly rotate the circular cutting arrangement 12. Thus, in FIG. 2, for example, a surgeon/user can hold the instrument 10 via the finger holders 16 (or via any other feature(s)/part(s)), and can then, with the circular cutting arrangement introduced to a cut point, and preferably with mild downward/inward force, twist/rotate the instrument, thus rotating the circular cutting arrangement. (Twisting is a type of rotating). Preferably the circular cutting arrangement is rotated in one direction. However, the circular cutting arrangement may be rotated back and forth, in different directions. (Thus a full rotation may not be required).

Preferably the circular cutting arrangement 12 has a diameter (and/or creates a circular cut) of approximately 3 mm. More preferably the circular cutting arrangement 16 has a diameter (and/or creates a circular cut) of approximately 2 mm. It will be known to those with skill in the art that a cut to fascia of approximately 3 mm is known to heal well, with little or no adverse effects. Having the circular cut to fascia be greater than 3 mm may increase likelihood that the cut will not heal so well (especially if tissue is being removed by the cut). Thus the cut made by the circular cutting arrangement is preferably 3 mm, or less. More preferably, the cut made by the circular cutting arrangement is preferably 2 mm, or less. (However, it is feasible the cut made by the circular cutting arrangement is more than 3 mm). Thus preferably the cut made by the circular cutting arrangement is less than 5 mm. Thus preferably the circular cutting arrangement 16 is dimensioned as such.

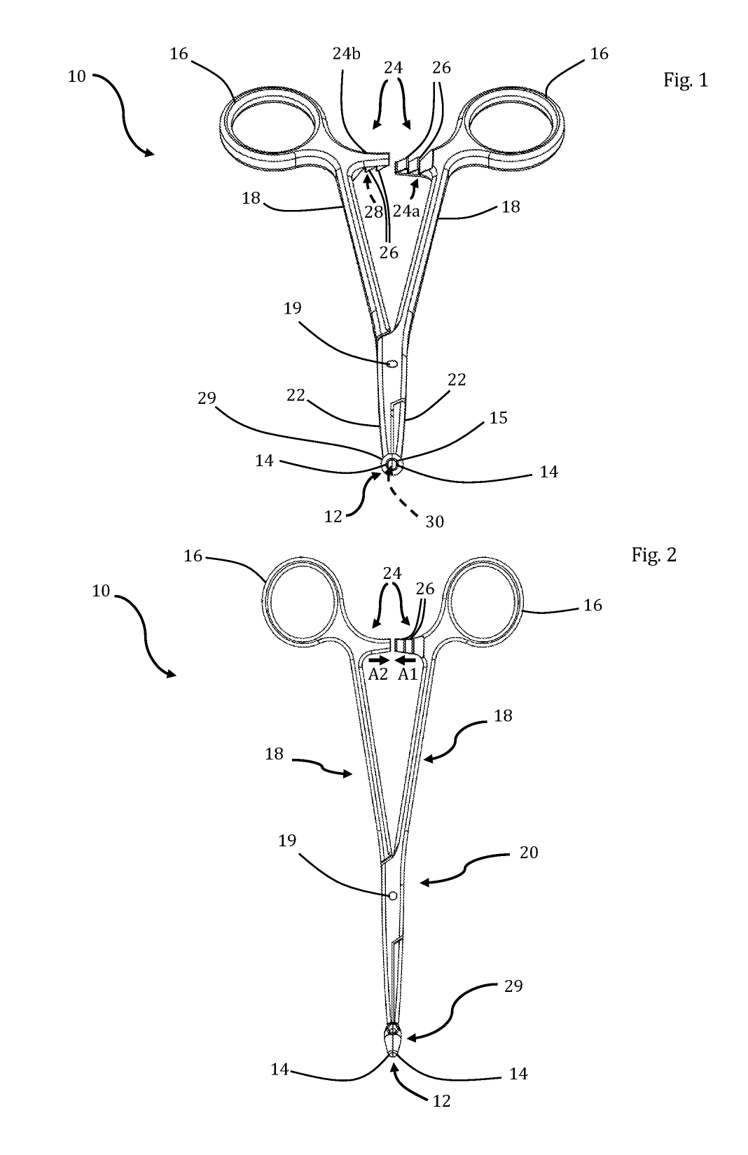

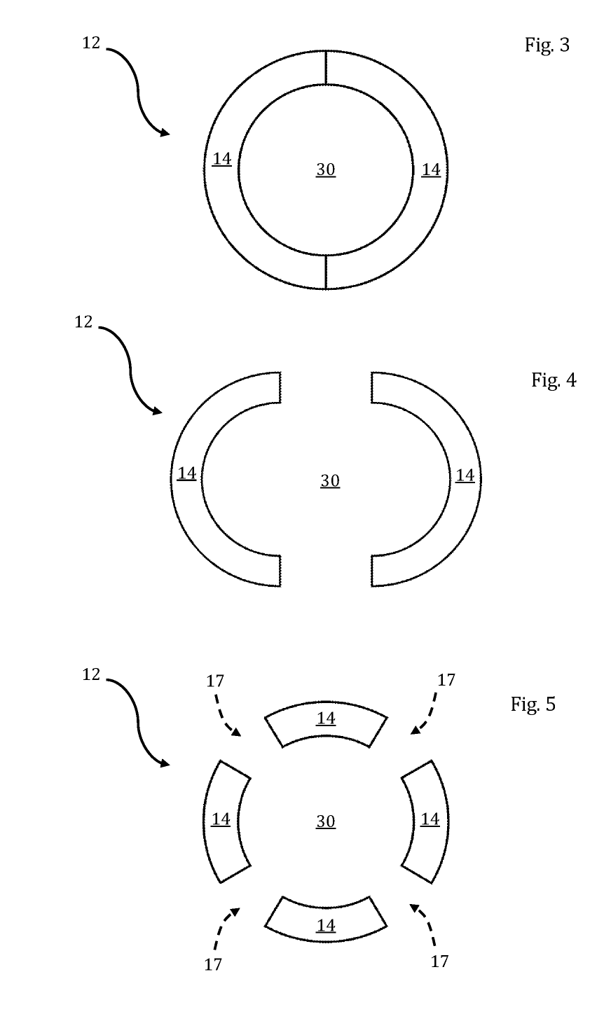

Preferably the circular cutting arrangement comprises a plurality of cut parts 14, (although it is feasible it may be formed of one part). In the examples of FIGS. 1 to 4 and FIGS. 6 and 7, the circular cutting arrangement comprises two cut parts. (Thus these are examples of the circular cutting arrangement comprising a plurality of cut parts 14). In the said examples, the cut parts are each substantially semi-circular. This is best shown in FIG. 3, where the example substantially semi-circular cut parts are shown closed together, thus forming a circular shape, and is also best shown in FIG. 4, where the example substantially semi-circular cut parts are shown opened. This may be useful, for reasons which will be explained.

In FIG. 5, the circular cutting arrangement is shown comprising a plurality of cut parts. In the example of FIG. 5, the circular cutting arrangement is shown comprising four cut parts. The circular cutting arrangement may be configured in any way, with any amount of cut parts and/or cut surfaces.

All the examples shown show a circular cutting surface 15. In the examples shown, most clearly shown/denoted in FIGS. 3 to 5, each cut part (if a plurality of cut parts are provided) comprises a cut surface. The sharpness of the cut surface is not shown/denoted in FIGS. 3 to 5. Nevertheless, preferably the cut surface(s) are relatively sharp. In the examples shown, (for example, in FIG. 1), the example circular cutting arrangement functions as a circular razor. As shown in FIG. 5, the circular cutting surface may comprise gap(s).

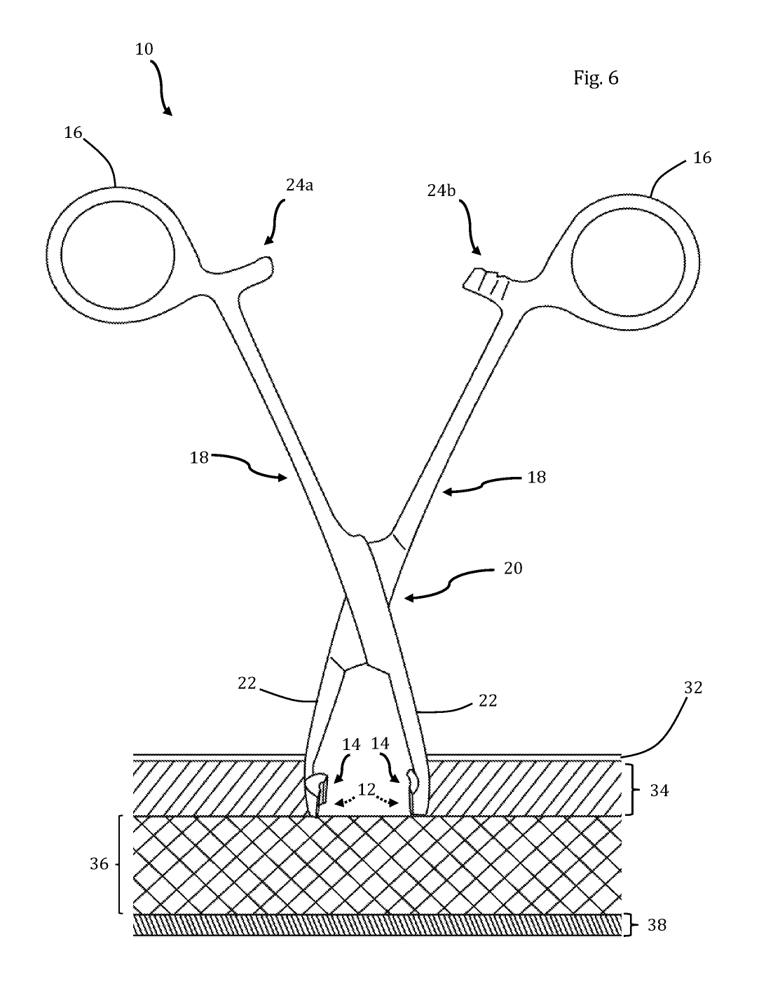

Preferably the instrument 10 comprises a cut-spreading arrangement, for spreading a cut made by the circular cutting arrangement. This may be useful, for example, to make it easier to insert laparoscopy device(s), instrument(s), etc through the cut made by the circular cutting arrangement. A cut-spreading arrangement, for spreading a cut made by the circular cutting arrangement, may be particularly useful to spread fascia cut by the circular cutting arrangement, (as will be shown in FIG. 6, for example). The cut-spreading arrangement, for spreading a cut made by the circular cutting arrangement, may also facilitate spreading (and thus may be used to spread) other tissue(s), which may, or may not, have been cut by the circular cutting arrangement. Thus the arrangement is not limited only to being able to spread fascia. For example (as shown in FIG. 6, by way of example), the arrangement may be used to spread a cut to skin (which cut to skin may, or may not, have been made by the instrument/circular cutting arrangement—the cut to skin may, for example, have been made by a scalpel, and not by the instrument 10). Similarly, the cut-spreading arrangement, for spreading a cut made by the circular cutting arrangement, may be used to spread muscle tissue(s), which may, or may not, have been cut by the circular cutting arrangement (and may, or may not, have been cut at all). For example, the muscle(s) may not have been cut at all, and simply may be pushed aside by the cut-spreading arrangement, for spreading a cut made by the circular cutting arrangement.

The drawings, (probably best shown in FIG. 6), thus show an embodiment(s) of an arrangement operable to spread a cut made by the circular cutting arrangement. The drawings, (probably best shown in FIG. 6), thus show an embodiment(s) of an arrangement configured to facilitate spreading a cut made by the circular cutting arrangement. Such example(s) are provided by way of example only.

In the examples shown, the cut is openable/spreadable via a portion or a whole of the circular cutting arrangement being movable. (However, this is just one example of an arrangement to facilitate spreading a cut made by the circular cutting arrangement, taken by way of example only). More preferably, as shown, preferably a portion or a whole of the circular cutting arrangement is openable. Thus, in examples where the circular cutting arrangement comprises a plurality of cut parts 14, preferably at least one or all of the plurality of cut parts are movable (more preferably, openable), to spread a cut made by the circular cutting arrangement. (This will be shown most clearly in FIG. 6). In examples where the circular cutting arrangement comprises a plurality of cut parts 14, preferably at least one or all of the plurality of cut parts are movable outwardly (more preferably, openable outwardly), to spread a cut made by the circular cutting arrangement. (This will be shown most clearly in FIG. 6).

Thus, preferably a portion or a whole of the circular cutting arrangement is movable (as shown in the examples). More preferably, preferably a portion or a whole of the circular cutting arrangement is openable (as shown in the examples). Broadly put, preferably the circular cutting arrangement is partially or wholly movable. More preferably, preferably the circular cutting arrangement is partially or wholly openable. This is shown in the example embodiments shown, and best shown in the comparison of FIG. 3, (where the example circular cutting arrangement is shown closed), and FIG. 4, (where the example circular cutting arrangement is shown opened). Thus, in FIG. 3 and FIG. 4, an example is shown where there are a plurality of cut parts 14, (in the example, the cut parts 14 being substantially semi-circular), and where they can be opened up from one another (shown in FIG. 4). They can also be closed together (shown in FIG. 3). This may also be the case with the example of FIG. 5, for example, where it is feasible any or all of the cut parts 14 may be opened outwards.

Preferably, a portion or a whole of the circular cutting arrangement is movable, to increase its distance away from a central point of the circular cutting arrangement. This is shown most clearly in FIGS. 3 to 5, and in particular, shown in the example of FIG. 3 and FIG. 4. In the embodiments shown (shown most clearly in FIG. 3 and FIG. 4, where the circular cutting arrangement is shown moved from a closed position, to an open position), the circular cutting arrangement comprises a plurality of cut parts, and it is clear, in FIG. 4, that the cut parts have both, in the example, moved, with their position now increased in distance away from the central point of the circular cutting arrangement. Thus the cuts parts, in FIG. 3, are closer to the central point of the circular cutting arrangement than the cuts parts are, in FIG. 4.

The examples show examples of a portion of the circular cutting arrangement being movable in position, relative to an(y) other portion of the circular cutting arrangement. The examples show examples of the circular cutting arrangement comprising a plurality of cut parts, and at least one cut part being movable in position, relative to an(y) other cut part of the circular cutting arrangement. This is best shown, for example, in FIG. 4, where it can be seen, in FIG. 4, that the cut parts have moved in position, relative to each other (compared to the (closed) position shown in FIG. 3). In the example, thus a portion has moved in position, relative to a central point of the circular cutting arrangement. In the example, thus at least one cut part has moved in position, relative to a central point of the circular cutting arrangement.

FIG. 5 shows that the circular cutting arrangement may, feasibly, comprise gap(s) 17. However, preferably the circular cutting arrangement does not comprise gap(s). (An example of the circular cutting arrangement not comprising gap(s) is best shown in FIG. 3, for example. FIG. 4 is also an example that does not comprise gap(s)—the cut parts are simply opened up from one another).

Thus the circular cutting arrangement 12 does not have to form a full circle, which is made clear by the example of FIG. 5. Nevertheless, it is clearly an example of a circular cutting arrangement, and is clearly configured to carry out a circular cut.

The position of the cuts parts 14 in the example of FIG. 3 may be termed a ‘closed position’. The position of the cuts parts 14 in the example of FIG. 4 may be termed an ‘open position’. Thus FIGS. 1, 2, 3, and 5 show example embodiments of the circular cutting arrangement in a closed position; FIG. 4 and FIG. 6 show example embodiments of the circular cutting arrangement in an open position.

The ability to open up the circular cutting arrangement can be useful to open up/spread a cut, once the cut has been made by the circular cutting arrangement. This can be useful to help make space to enter various device(s) and/or instrument(s) through the cut, to carry out a laparoscopy.

Preferably the instrument 10 comprises an arrangement to facilitate moving (preferably opening) the plurality of cut parts of the circular cutting arrangement. (Most preferably, the instrument 10 comprises an arrangement to facilitate opening and closing the plurality of cut parts of the circular cutting arrangement). In the example embodiments shown, the example arrangement to facilitate moving (preferably opening) the plurality of cut parts of the circular cutting arrangement (which also, in the example, facilitates closing the plurality of cut parts of the circular cutting arrangement), is/comprises an opposing arrangement. More specifically, in the examples shown, the arrangement to facilitate moving (preferably opening) the plurality of cut parts of the circular cutting arrangement is provided by way of a scissor arrangement. A scissor arrangement is an example of wherein the opposing arrangement comprises opposing handle parts. (Each handle part, in the example, comprises a finger holder 16. However, handle parts are not limited to having to comprise a finger holder). The example scissor arrangement is shown comprising opposing limbs 18. The example scissor arrangement is also shown comprising finger holders 16.

(Thus the cut-spreading arrangement, for spreading a cut made by the circular cutting arrangement may, or may not, comprise an opposing arrangement, in no way limited to the shown examples. Thus the cut-spreading arrangement, for spreading a cut made by the circular cutting arrangement may, or may not, comprise a scissor arrangement, in no way limited to the shown examples).

In the example, the limbs are able to cross at what could be termed a crossing arrangement 20. The limb(s) 18, in the example, culminate at (and/or lead to) the circular cutting arrangement 12 and/or at a cut part 14 of the circular cutting arrangement. This is simply one example of providing an instrument 10 comprising a circular cutting arrangement 12.

In the example, the opposing arrangement and/or scissor arrangement and/or limb(s) and/or crossing arrangement is hinged. A hinge point 19 and/or hinge screw 19 is shown, in FIG. 1 and FIG. 2, shown by way of example only.

The example(s) are shown comprising a portion(s) 22 that culminate(s) in (and/or lead to) the circular cutting arrangement and/or a cut part of the circular cutting arrangement. The portions 22, in the example, that culminate in the circular cutting arrangement and/or a cut part of the circular cutting arrangement (best shown in FIG. 6) may be referred to, in the example(s), as an arm element. An arm element and/or portion that culminates in the circular cutting arrangement and/or a cut part of the circular cutting arrangement is not limited to being provided in a scissor arrangement, and is not limited to being provided in the examples shown, and may be provided in any embodiment. An arm element(s) and/or portion(s) 22 is not limited to being provided in an opposing arrangement, and is not limited to being provided in an arrangement that comprises a longer limb(s)′, for example. An arm element(s) 22 and/or portion(s) may be provided in any embodiment. A portion(s) 22 (and/or arm element(s)) may be any shape or size. Preferably the or any portion 22 (and/or arm element) is elongate. The or an arm element(s), in the shown example(s) is a portion(s) which culminates in a cut part and/or cutting arrangement. Thus the arm element(s) shown, in the examples, are simply embodiments of portions which, in the shown embodiment(s), culminate in (and/or lead to) a cut part and/or cutting arrangement.

(Thus, broadly put, preferably the instrument is configured to be able to spread a cut made by the circular cutting arrangement).

(In the example(s) shown, the instrument is operable to spread a cut made to fascia by the instrument).

In the example, (shown best in FIG. 2), there is shown a portion 29. In the example, the portion 29 is above and/or extends from the circular cutting arrangement. In the example, the circular cutting arrangement is formed from the portion 29 and is, in the example, a sharpened part of the portion 29. Thus, in the example embodiments, the circular cutting arrangement is manufactured by sharpening a part (preferably an end) of the portion 29. (This is simple shown by way of example only). In the example, at least a portion or a whole of the portion 29 has a greater diameter and/or width than the circular cutting arrangement. In the example, at least a portion or a whole of the portion 29 has a greater diameter and/or width than the portion(s) 22 and/or arm element(s) and/or body from which the portion 29 directly leads. (This is best shown in FIG. 2, where it can be seen the instrument is thinner just above the portion 29). In the example, the portion 29 is generally cylindrical. In the example, the portion 29 comprises a hollow (and/or is hollow). More specifically, (as shown in the example), the portion 29 preferably comprises a circular hollow. This is best shown, perhaps, in FIG. 6, wherein the circular cutting arrangement is shown in an open position. Thus the portion 29 is preferably partially or wholly hollow. Thus it is shown (shown clearly in FIG. 6) the portion 29 itself, in the example, comprises a plurality of portion parts. In the example(s), (and shown most clearly in FIG. 6), the portion 29 comprises a first substantially semi-circular portion part, and a second substantially semi-circular portion part. (Thus, there may be provided a portion part for each cut part). In the example, each portion part culminates in (and/or leads to) a cutting part 14. In the example, the cutting parts are themselves both substantially semi-circular. The preferably circular hollow of the portion 29 is clearly visible in the open position of FIG. 6. Thus preferably the circular cutting arrangement, and the portion 29 (if one is provided) are both hollow. Preferably both have a hollow within them. Preferably the hollow of both is circular. Thus preferably there is a portion that extends from (and/or is above) the circular cutting arrangement, wherein preferably both the circular cutting arrangement and the portion are partially or wholly hollow and/or have a hollow within them. Thus the hollow preferably continues up the portion 29 (as shown in FIG. 6, for example). This forms, in the example, a hollow and extended arrangement. This can be beneficial for hollow cutting.

Preferably, the instrument 10 comprises a locking arrangement 24, to lock the circular cutting arrangement in a closed position. In the examples shown, the locking arrangement comprises a first arrangement, and a second arrangement, that in some way interact, to lock the circular cutting arrangement in a closed position. In the examples shown, the locking arrangement comprises an interlocking arrangement. The interlocking arrangement comprises, in the example, a first arrangement 24a, and a second arrangement 24b. The first arrangement 24a, in the example, comprises teeth 26. It may comprise at least one tooth 26, for example. The second arrangement 24b, in the example, comprises a corresponding surface 28 that can receive the at least one tooth 26. This allows the first and the second arrangement to be interlocked together. In the example, the corresponding surface 28 also comprises at least one tooth 26. In the example, the corresponding surface 28 of the second arrangement 24b also comprises teeth. Thus, broadly, put, in the example, the first arrangement and second arrangement both comprises at least one tooth. This, in the example, facilitates the first arrangement and second arrangement interlocking together. In the example, the second arrangement comprises the same amount of teeth as the first arrangement. In the example, the corresponding surface 28 comprises the same amount of teeth as the first arrangement.

In the example embodiment shown, the first arrangement 24a and second arrangement 24b are identical to each other in design and structure. (In FIG. 1, a dashed line is used to point to the corresponding surface 28, to denote it is on the other side of the second arrangement 24b). In an example wherein an interlocking arrangement is provided, as shown, the interlocking arrangement may comprise at least one tooth. More preferably, such an example embodiment of an interlocking arrangement comprises teeth. This includes within its scope, for example, a first arrangement 24a comprising teeth (or at least one tooth); and/or a second arrangement 24b comprising teeth (or at least one tooth). As shown, preferably an interlocking arrangement is provided wherein both a first arrangement and a second arrangement are provided, and wherein both comprise teeth.

Thus it can be seen, then, for example, (best shown with reference to FIG. 2), that if, in the example, the handle parts are pulled together tightly, the first arrangement and second arrangement can be locked together (in this case, by being interlocked). This, in the example, locks the circular cutting arrangement in a closed position. In the example of FIG. 2, some force may be needed to push together handle parts and/or the first and second arrangements, in order to interlock the first and second arrangements. This may lead to the circular cutting arrangement being extremely tightly closed together. This may be beneficial. Thus, whilst the circular cutting arrangement is shown in a closed position in FIG. 2, if the locking arrangement 24 is locked together, it will be obvious this may extremely tightly lock the circular cutting arrangement in a closed position. (Arrows A1 and A2 in FIG. 2 denote direction of how the example arrangements 24a and 24b can be pulled together, to then interlock. In the example, this would tightly lock the circular cutting arrangement in a closed position. (The material(s) the instrument is constructed of (eg stainless steel) may, or may not, have slight flexibility to allow this)).

Interlocking is just one way that a first arrangement and a second arrangement may interact, in order to lock the circular cutting arrangement in a closed position. Use of a first and a second arrangement is itself just one way of providing a locking arrangement. Any arrangement may be provided.

In the examples shown, the first arrangement 24a is provided on a first limb and/or handle part; and the second arrangement is provided on a second limb and/or handle part. More generally put, in the example, the first arrangement 24a is provided on a first opposing part; and the second arrangement 24b is provided on a second opposing part.

In the examples shown, the first arrangement 24a and the second arrangement 24b are both elongate in shape. This is shown by way of example only.

Preferably the circular cutting arrangement is partially or wholly hollow. The examples shown in the Figures all show examples of the circular cutting arrangement being partially or wholly hollow. In the examples shown, the circular cutting arrangement is wholly hollow. This is best shown in FIGS. 3, 4, 5, and is also shown well in FIG. 6. The examples shown (shown by way of example only) show there being a hollow 30 within the circular cutting arrangement, (most clearly denoted in FIG. 1 and FIGS. 3, 4 and 5).

Preferably the instrument is partially or wholly metallic, although it may be constructed of any material(s). Most preferably, the instrument is constructed partially or wholly of stainless steel.

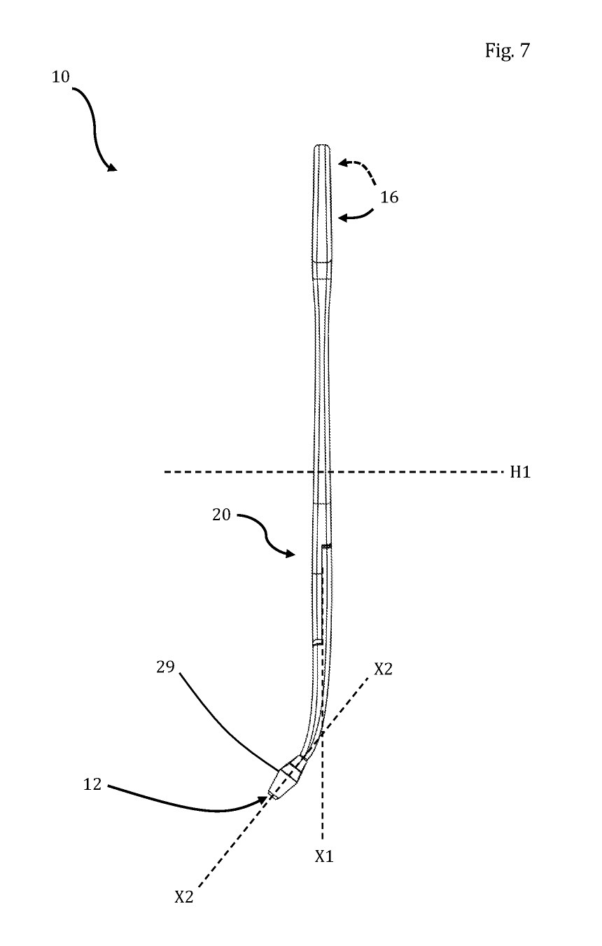

There is shown in FIG. 7 an example of the instrument 10 wherein a portion or a whole of the instrument is curved. In the example, a portion or a whole of a half of the instrument is curved. In the example, a portion or a whole of a half of the instrument is curved, the half being the half that includes the circular cutting arrangement. Dashed line H1 cuts through the instrument, showing it divided in halves. From viewer perspective of FIG. 7, the bottom/lower half includes the circular cutting arrangement 12. This, therefore, is the half that includes the circular cutting arrangement. Thus, in the example, a portion or a whole of a half of the instrument is curved, the half being the half that includes the circular cutting arrangement.

More broadly put, preferably a portion or a whole of a half of the instrument deviates in angle from any other portion of, or most of, the instrument. More specifically, preferably (as shown in FIG. 7), a portion or a whole of a half of the instrument deviates in angle from a portion of, or most of, the instrument, the half being the half that includes the circular cutting arrangement. Thus it is not limited to deviating in angle via being curved; for example, rather than the example of FIG. 7 being curved, it may, instead, be angular, and angularly change/deviate in angle, in a straight line, rather than by being curved. Therefore curving is not the only way for a portion of the instrument to deviate in angle.

Thus, as shown clearly in the example of FIG. 7, preferably the circular cutting arrangement differs in direction from the rest of the instrument. This is clearly shown, in the example of FIG. 7, by axis X1, which shows a directional axis (ie direction) of most of the instrument. Axis X2 then shows directional axis of the circular cutting arrangement. Thus it shows/denotes/represents direction that the circular cutting arrangement is pointing. It is clear the circular cutting arrangement is pointing in a different direction to axis X1.

Thus, more broadly put, preferably a directional axis of the circular cutting arrangement is different from a portion of, or most of, the instrument. This may be the case for any embodiment of the instrument, and is not limited to the examples shown. Thus it is not limited to examples that have handle parts and/or an opposing arrangement, and/or a crossing arrangement, etc. The directional axis of the circular cutting arrangement may be different from a portion of, or most of, the instrument, in any embodiment of the instrument, and may be achieved in any way.

(It should be noted, this feature (of curving and/or deviation in angle) is also present and/or shown in the examples of FIG. 1, FIG. 2, (and FIG. 6, to some extent), but is not so immediately obvious as in FIG. 7, due to the view angle of FIG. 1, FIG. 2, and FIG. 6. It can, however, be seen that the circular cutting arrangement is pointing up, toward the viewer, in FIG. 1. This is due to the curve (and therefore change in directional axis of the circular cutting arrangement), as shown more clearly in FIG. 7, from the side profile view).

Preferably, as shown in the examples, the circular cutting arrangement is provided at an end of the instrument.

### In Use

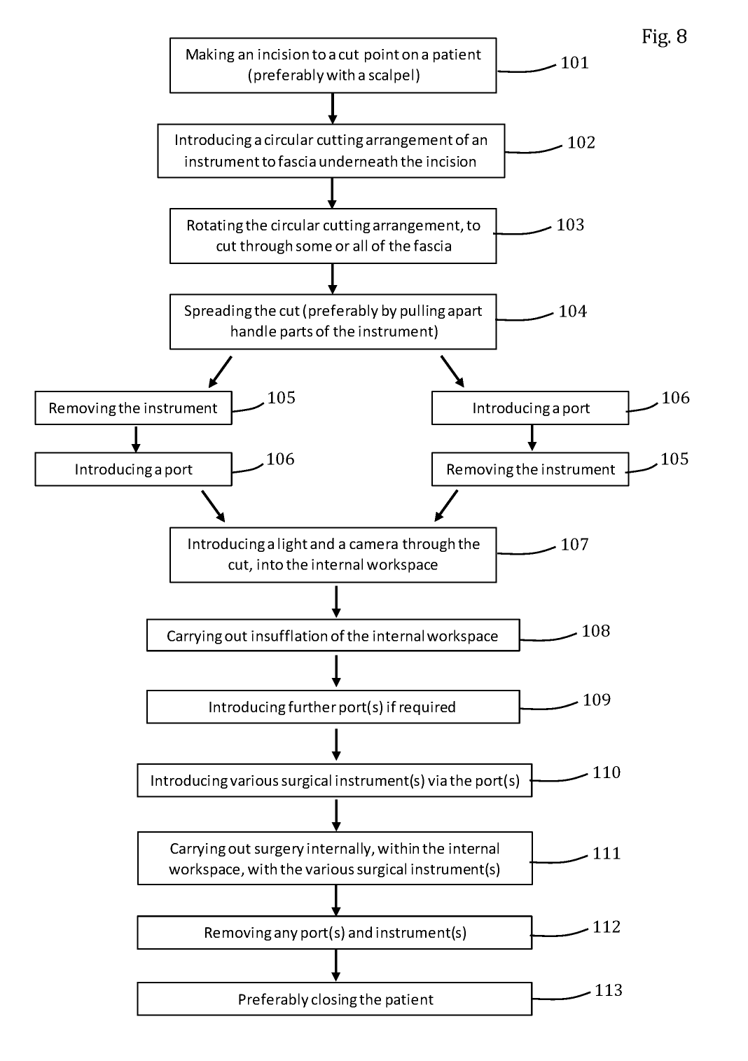

An example(s) will now be described in use, described by way of example only, referring to a particularly preferred embodiment (eg as shown, and with particular relevance to what is shown in FIG. 6, which shows an embodiment of the instrument, in use), in no way limiting a scope of the invention. (The example will be described in a way relevant to the flowchart of FIG. 8). The example will be described, with reference to a method for carrying out laparoscopic surgery.

Thus, referring in use (by way of example) to a particularly preferred example, preferably an incision is made to skin of a patient. This is preferably made by a scalpel. (This is generally referenced in box/step 101 of FIG. 8). It is feasible the incision to the skin is itself made by the circular cutting arrangement of the instrument. However, as will be known to those with skill in the art, if a circular cut is made that removes skin of a patient, it can or will increase the chance of infection. Therefore it is preferable that an initial cut/incision is made without removing skin. Therefore preferably a cut is initially made, with a scalpel, for example. The incision made (eg by a scalpel) is preferably 3 mm-5 mm in length.

The circular cutting arrangement is then preferably introduced to fascia underneath the skin, at the cut point. (This is generally referenced in box/step 102 of FIG. 8). (Preferably the cutting arrangement is locked in a closed position, before cutting begins. In the examples shown, this can be done by interlocking the first arrangement 24a and second arrangement 24b). (The cut point is here defined as a point at which a cut is to be made. As stated, an incision (eg by a scalpel) may, or may not, have already been made at the cut point).

Preferably some downward/inward pressure is applied (eg against the abdominal wall), and the circular cutting arrangement is rotated, to cut through the fascia. (This is generally referenced in box/step 103 of FIG. 8). In the example(s) shown, circular rotation is achieved by the surgeon holding the instrument in a scissor like fashion, with finger(s) and/or thumb in finger holders of the instrument. A twisting motion is then used, to rotate the instrument and the circular cutting arrangement, in such embodiment. In the example(s) shown, for example, the surgeon/user may hold the instrument in a scissor like fashion, via the example finger holders, and may implement a twisting motion, back and forth from clockwise to counter-clockwise (or vice versa), for example, to make a cut.

(If the circular cutting arrangement is itself used also to initially make the incision/cut to the skin (rather than a scalpel, for example), then it will be obvious that the cutting, rotation, inward pressure, etc may start at that point, rather than only when the circular cutting arrangement reaches the fascia, and that the circular cutting arrangement may therefore not need to be separately ‘introduced’ to the fascia, as it would be after a skin cut made by a scalpel, for example. Nevertheless, even in such a case (where the circular cutting arrangement is used to make the initial skin cut), this nevertheless falls within a scope of the circular cutting arrangement being introduced to a cut point. Thus whether the circular cutting arrangement is introduced to fascia, with an initial incision in the skin having been made (eg by a scalpel), or whether the circular cutting arrangement is introduced to the skin, and is used to cut through the skin, and on through fascia, both of these fall within a scope of the circular cutting arrangement being introduced to a/the cut point).

It will be well known to laparoscopy surgeons that significant resistance can be encountered when cutting through the fascia, and that, when the fascia is fully penetrated, there is often a clear and obvious release of such resistance. Preferably the surgeon continues to rotate the circular cutting arrangement until the fascia is fully penetrated through. It is thought this method of penetrating through the fascia makes it extremely less likely that internal organs and/or structures will be damaged by the instrument ‘bursting through’ the fascia.

Preferably all the fascia is cut through, using the circular cutting arrangement. Although it is feasible the instrument (and circular cutting arrangement) may continue to be used to penetrate through further tissue(s), (such as muscle tissue and/or parietal peritoneum tissue and/or fatty tissue), preferably the instrument (and circular cutting arrangement) is not used to cut such tissue(s). It will be known to surgeons that often muscle(s) can be moved aside, to allow instruments to be inserted. Furthermore, parietal peritoneum tissue and/or fatty tissue tend not to be difficult to insert instrument(s) through. Therefore they may not need to be cut by the cutting arrangement. Therefore the instrument may, or may not, cut through any further tissue(s). Therefore muscle and/or parietal peritoneum tissue is preferably not cut by the cutting arrangement.

When/if the fascia is fully penetrated through, a portion or a whole of the cutting arrangement may go fully through the fascia, at the final point of penetration, when the cutting arrangement finally gets through the fascia. Thus, for example, just a portion of the circular cutting arrangement may penetrate through the final part of the fascia. Preferably the whole circular cutting arrangement penetrates and gets through the fascia. Nevertheless, once the penetration has occurred, even if a portion (rather than a whole) of the cutting arrangement gets through, this may be all that's required. At that point, the cut may then be spread (whether a portion or a whole of the cutting arrangement has gone through the fascia). Whether a portion, or a whole, of the circular cutting arrangement penetrates through the final part of the fascia, preferably the fascia that has been cut (eg a circular tube of fascia, which may have ascended up a hollow of the circular cutting arrangement, during the cut) remains intact, in that it preferably (or may) remain attached to other fascia and/or peritoneum and/or any other tissue. Thus it preferably stays intact, and within the patient.

Preferably the cut made by the circular cutting arrangement is now spread. (This is generally referenced in box/step 104 of FIG. 8). (If the circular cutting arrangement has been locked in a closed position, preferably it is unlocked, before the cut is spread, so that it can be opened, to spread the cut, for embodiments where this is the or a method for how to spread the cut). In example embodiments shown, (wherein the circular cutting arrangement comprises a plurality of cut parts), this is preferably done by virtue of opening at least one or all of the plurality of cut parts of the circular cutting arrangement (as shown well in FIG. 6). (The spreading of the cut made by the instrument may, or may not, tear fascia tissue. However, if any tearing occurs, it is not thought such tearing causes any significant damage to the patient's health and/or to the result of the or a laparoscopy surgery). Thus, preferably, fascia, having been cut, is spread.

In FIG. 6, the example shows fascia 34 being spread by the instrument. A skin 32 layer of the patient is shown. A representative layer is shown to denote muscle tissue 36. A representative layer is shown to denote what may be parietal peritoneum 38. It will be known that these layers (and the Figure) are shown representationally, showing a representational cross section of tissues inside the patient, to better denote an example of how the instrument may be used. Such tissues tend to be less linear in terms of how they are layered and/or positioned. Furthermore, there may be further tissue(s) present, such as fatty tissue, for example. Thus FIG. 6 is provided representationally, and to provide better understanding of how an example embodiment of the instrument may be used, and may be useful. (The representational tissues of the patient are shown cross-sectionally, but the instrument is not shown cross-sectionally).

In the example of FIG. 6, it can be seen that an incision to the skin has been made (preferably by a scalpel), and the instrument is then shown, in this example having cut through all (or at least a part of) the fascia, now spreading the fascia. As stated, it will be known to surgeons that often muscle(s) 36 can be moved aside, to allow instruments to be inserted. As stated, parietal peritoneum tissue 38 and/or fatty tissue tend not to be difficult to insert instrument(s) through. Therefore they may not need to be cut with the instrument 10. Thus, in the example of FIG. 6, the instrument 10 has not been used to cut through muscle, or parietal peritoneum, etc. (However, it is feasible the instrument may be used to cut through any such tissue(s) also).

(FIG. 6 shows an example of wherein a portion or a whole of a part of the instrument that is inside the cut is able to spread and/or expand outwards, to spread a cut. (This is shown, in action, in FIG. 6)).

Preferably a port is now introduced/inserted. (This is generally referenced in box/step 106 of FIG. 8). (The term ‘port’ will be well known to those with skill in the art, and is a unit used regularly (or in all) laparoscopy surgery). The port may or may not be inserted through the cut whilst the instrument is still in place (ie whilst a portion or a whole of the instrument 10 and/or circular cutting arrangement is still within the cut that has been made and/or inside the body of the patient). Thus the instrument comprising the circular cutting arrangement may still be in place at this point. Alternatively, the instrument comprising the circular cutting arrangement may be removed from the cut/patient, (generally referenced in box/step 105 of FIG. 8), and the port may be inserted and/or positioned, after the instrument has been removed. Thus the instrument comprising the circular cutting arrangement may be removed at this point, or kept in, eg temporarily, to guide a port through the cut. (Thus the flowchart of FIG. 8 denotes that box/step 105 may occur before box/step 106, or may occur after). (As stated, it may be possible to introduce the port(s) without cutting any muscle (eg by moving the muscle(s) aside). It is also possible that the port(s) may be pushed through tissue(s). Thus the port(s) may, itself, penetrate (and/or be pushed) through tissue(s)).

Preferably an instrument is then inserted through the port and through the cut, with the instrument tending to include a light and a camera. Thus preferably a light is inserted through the cut. Preferably a camera is inserted through the cut. (This is generally referenced in box/step 107 of FIG. 8). Preferably insufflation of the internal area of the body, where the laparoscopy is going to occur (eg the abdominal cavity, for example) is carried out. (This is generally referenced in box/step 108 of FIG. 8). (Insufflation is often carried out to create extra space in the workspace inside the patient's body, where the surgery will occur). CO2 is often used for insufflation, although any gas(es) may be used. This (insufflation) is often done with the same instrument that includes/comprises a light and/or camera. Thus this may be carried out with the same instrument that comprises the light and/or camera and may be inserted into the body through the same port. At this point, the surgeon has successfully achieved inserting a light source and a camera, and may use insufflation to make space for the laparoscopy that is to come.

Further port(s) may now be introduced/inserted into the cut (and/or any further cut(s)). Further cut(s) may be made by the instrument. Thus the instrument (and circular cutting arrangement) may be used to make further cut(s) to the patient. Port(s) (eg, further port(s) may be introduced to any further cut(s). Thus the instrument may be used to make a plurality of cuts to the patients. Thus there may be a plurality of cut points. It is not uncommon in laparoscopic surgery for a plurality of cuts to be made to a patient during a procedure, and for there thus to be a plurality of cut points (and/or port(s)). (There tends to be one port used per cut). (This is generally referenced in box/step 109 of FIG. 8). These may be used, for example, for surgical instrument(s), to carry out the laparoscopy. Thus, via the first port, or further port(s), (or via any means under the Sun), surgical instrument(s) to carry out the laparoscopy may now be inserted through the cut. (This is generally referenced in box/step 110 of FIG. 8). The surgeon then carries out the laparoscopy surgery. (This is generally referenced in box/step 111 of FIG. 8). Once the surgeon has completely the surgery, the port(s) and/or instruments are removed from the cut and/or the patient. (This is generally referenced in box/step 112 of FIG. 8). Preferably the skin/patient is then closed (eg via suturing and/or stitching). (This is generally referenced in box/step 113 of FIG. 8). The procedure is now complete.

(It is possible it is not required to close the patient and/or skin of the patient. It will be known that, if the cut made by the instrument is relatively small (eg 5 mm or less) that it is possible the patient may heal, without needing closing of any cut. However, preferably the skin is closed (eg by suturing). Furthermore, the fascia may be closed (eg by suturing. (This may include the cut, and any further tearing and/or spreading that has occurred from spreading the fascia and/or from any action during the procedure). Thus preferably the fascia and/or the skin of the patient is closed. It may be that both the skin, and the fascia, are separately closed (eg via being separately sutured). Thus the cut is preferably closed. This may be the case for any further cut(s) made (which may, or may not, have been made by the instrument. Thus it is feasible a plurality of cuts may be made by the instrument, and that the plurality of cuts may be closed. Thus the or any of the cut(s) may be closed. (The fascia and/or skin of such cut(s) may be closed). As stated, the cut to the skin may or may not have been made by the instrument.

(If there is any concern over infection, it is possible a skin cut(s) may be left open. In such a case, the fascia may, or may not, be closed (eg sutured)).

The circular portion of the fascia that is cut by the circular cutting arrangement preferably remains attached to the fascia and/or peritoneum and/or any other tissue(s), and therefore ‘intact’. In this way, it may remain part of the fascia/patient, and may retain blood flow. Thus it may remain in the patient, and may not need to be removed.

Thus, referring to a method for carrying out laparoscopic surgery, there may be provided a method for carrying out laparoscopic surgery, comprising: introducing a circular cutting arrangement to a cut point on a patient; and circularly rotating the circular cutting arrangement, to make a circular cut through fascia. The method may draw upon any disclosure and/or feature(s) of the instrument in the present application, and/or may draw upon any disclosure of the present application. (The or a method may be claimed as a method for cutting fascia. The or a method may be claimed as a method for cutting fascia for laparoscopy).

As stated, preferably an incision is made to skin of the patient, before circular cutting begins. Preferably, if such an incision is made, such an incision is made by a scalpel, although it could be made by any means under the Sun. Preferably the surgeon/user then rotates a portion or a whole of the instrument, to rotate the circular cutting arrangement.

Referring to the method, step(s) disclosed in the present application and/or in FIG. 8, where possible, are shown in chronological order. However, the or any step(s) are not limited to the order they are disclosed and/or shown and/or claimed, and may be carried out/provided in any order.

All the features of the instrument 10 (eg all the features as seen, for example, in the embodiments shown in FIG. 1, FIG. 2, and FIG. 6), may, if so required, be referred to and/or defined as an instrument (and/or device) body. The instrument body, (as shown in such examples), is preferably elongate.

The circular cutting arrangement 12 does not need to be a perfect circle. For example, the example circular cutting arrangement shown in FIG. 3 is not a perfect circle. Nevertheless, it is circular, and is a circular cutting arrangement. The term ‘substantially semi-circular’ is used in describing the shape of the cuts parts in the example embodiment(s) shown in many of the drawings (eg as shown/represented in FIG. 3 and FIG. 4, for example). It should be noted, the term ‘substantially’ is used here because, in a layman's mind, the term ‘semi-circle’ is often (or may be) perceived as meaning a ‘perfect’ half of a circular shape, (whether the circular shape is a perfect circle or not). The term ‘substantially’ simply denotes here that the cuts parts need not be ‘exact’ semi-circles; for example, if one cut part takes up 55% of the circular cutting arrangement, and the other cut part takes up 45%, nevertheless, this would be within a scope of the cuts parts being ‘substantially’ semi-circular. However, use of the term ‘substantially’ with reference to the term ‘semi-circular’ should in no way be seen as suggesting that the term ‘circular’ is extremely limited. The term ‘circular’ has some natural broadness to it, compared to how a term like ‘semi-circular’ may be perceived, and, as explained, the circular cutting arrangement does not need to be a perfect circle. Thus it is not thought the term ‘substantially’ is required, with reference to the term ‘circular’. Thus the fact that the term ‘substantially’ is used with reference to the term ‘semi-circular’, but is not used with reference to the term ‘circular’ should in no way be seen as an argument to suggest undue or unreasonable limitation on the term ‘circular’.

Whether the circular cutting arrangement is circularly rotatable a full circular rotation, or a part of a circular rotation, it is considered within a scope of being circularly rotatable.

The embodiments described above are provided by way of example only, and various other modifications will be apparent to persons skilled in the art without departing from the scope of the invention as defined in the appended claims.

### Broader and/or Different Invention(s) May be Claimed (and are Supported)

The appended claims define limited inventions. However, it should be recognized and understood that the disclosure of the present application includes a vast array of inventions, not limited to inventions set out in the appended claims and/or any statement(s) of invention.

For example, if the present disclosure of the present application (inclusive of drawing(s) and/or description) discloses features a to z, it should be recognized and understood that any invention may be claimed, comprising any feature(s) out of features a to z. Thus if the appended claim 1 defines the invention claimed as comprising essential features a, b, and c, it should be understood that an invention may be claimed comprising solely feature a, or solely feature b, or solely feature c, or any combination of features a, b, and c. Furthermore, it should be understood that an invention may be claimed comprising any of feature(s) d to z, whether or not also comprising any of features a, b, or c.

Furthermore, no feature disclosed is limited to only being set forth in a claim when used in conjunction with other particular feature(s) it is disclosed with in the specification, but may be claimed with any other feature or combination of features disclosed in the present application. Thus if a feature is disclosed ‘clustered’ with several other feature(s) when disclosed in the specification, the applicant(s) nevertheless reserves the right to ‘extract’ that feature(s) from the several other feature(s) it is disclosed with, and set it forth in a claim, combined with any other feature(s) disclosed in the present application, which other feature(s) may, or may not, also be ‘extracted’ from any other feature(s) they are clustered with in the disclosure of the present application. Thus any permutation/combination of features may be claimed for patent in a future claim and/or patent application. Thus the instrument may be claimed, for example, in an embodiment comprising a cut-spreading arrangement, wherein the cut-spreading arrangement comprises an opposing arrangement, (in no way limited to having to comprise other feature(s) (such as a plurality of cut parts, etc) that are shown in example embodiment drawings that are shown comprising the/an opposing arrangement.

The applicant(s) reserve the right to claim any invention (ie ‘thing’), comprising any feature, or combination of features, disclosed in the present application (inclusive of drawing(s) and/or description). This statement (and/or final appended claim), if so desired, should be seen as a statement of invention, stating any invention (ie ‘thing’), comprising any feature, or combination of features disclosed in the present application (in any permutation/combination). The applicant(s) reserves the right to claim any (such) invention (ie ‘thing’), and considers an objection by a patent office/examiner (stating that such an invention is not supported by/disclosed in the present application) to be in direct conflict with this statement of invention. Thank you to the relevant patent office/examiner for taking note of this. It is intended (or plausible) that such invention(s) may be claimed in a future application(s) which claims benefit of priority of the present application, or, for example, in future filed claims of the present application. The present disclosure of the present application supports such invention(s)/claim(s).

### Adjectival and Adverbial Use, in the Present Application, is Innately Optional

In the present application, adjectival definition of a noun in no way limits the ability to claim the noun, without the adjective. Thus if a ‘curved edge’ is disclosed, it should be understood that it is disclosed simply by way of example, as an embodiment of ‘an edge’, and that an invention may be claimed, comprising an ‘edge’, and not limited to comprising a ‘curved edge’, even if the only disclosure in the specification is of a ‘curved edge’. This goes for every single adjective example in the present application, and also applies to adverbs in the same way, with reference to how they limit a broader verb/action, which verb/action/characterizing feature may be included in a claim (and is supported), not limited by the adverb that further defines it. This also applies to ‘usage for’ definitions. Thus if an instrument/apparatus, for example, is described, with reference to use for a particular thing (eg a ‘pet cleaning apparatus’, (which is therefore a cleaning apparatus, for use on pets)), then a ‘cleaning apparatus’ may be claimed, not limited to being a ‘pet’ cleaning apparatus and/or not limited to being for use on pets, even if all disclosure in the patent application relates to a ‘pet cleaning apparatus’. This also applies for an instrument disclosed as being an instrument ‘for use in [a given surgery]’, for example, (or for a given ‘usage’). In such a case, the/an instrument may be claimed for use in any surgery (not limited to the given surgery/usage), and/or may be claimed, simply as an instrument, (not limited to being for use in surgery, (and/or not limited to the usage stated)).

### The Title of the Present Application does not Limit What May be Claimed

The title of the present application (and the claims presented) do not limit what may be claimed futurely, based upon (and supported by) the present application. For example, if the title is ‘Pet Cleaning Apparatus’, even if all disclosure in the patent application relates to a pet cleaning apparatus (as do the claims), nevertheless, a ‘cleaning apparatus’ may be claimed (not limited to being for pets), as it is clear a ‘pet cleaning apparatus’ is an embodiment of a ‘cleaning apparatus’. As stated previously, in the present application, adjectival definition of a noun in no way limits the ability to claim the noun, without the adjective. This also applies to the title. Furthermore, an invention may be claimed comprising any feature, or combination of features, disclosed in the present application.

### Feature(s) Shown in the Drawings May be Combined to Form an Invention

Any feature(s) or combination of feature(s) shown in any drawing(s) may be combined with any other feature(s) or combination of feature(s) shown in any other drawing(s), to form an invention, which may be claimed. This may be the case for any embodiment shown in any drawing(s), and applicant(s) reserves the right to claim any such invention(s). Furthermore, such feature(s) may, of course, be combined with any other feature(s) and/or disclosure of the present application, to form an invention(s), which may be claimed. Such an invention(s) may be claimed in a future application(s) which claims benefit of priority of the present application, or, for example, in future filed claims of the present application. The present disclosure of the present application supports such invention(s)/claim(s).

Thus, in line with the above statement(s), a broad statement of invention is herein included, of: an instrument, comprising an instrument body. All other feature(s) and/or combination of feature(s) disclosed in the present application (eg spreading of the cut) are optional with regards to this broad statement of invention. Furthermore, any feature (not limited to being provided with/clustered with any other feature(s) disclosed in the present application (even when disclosed together ‘essentially’ with regard to any other (statement of) invention in the present application)) may be claimed in dependent fashion with regard to this broad statement of invention, and are deemed preferable and/or optional feature(s). This broad statement of invention should in no way affect searching and examining of any more narrow appended claims, and in no way affects the scope of such appended claims, which are likely to be far narrower in scope than the broad statement of invention provided in this paragraph. According to this aspect, the instrument may, or may not, be for use in laparoscopic surgery. The instrument may, or may not, be for use in surgery. The instrument may comprise a cutting arrangement. The cutting arrangement may, or may not, be a circular cutting arrangement. The cutting arrangement may, or may not, be for circular cutting. The instrument may, or may not, be for cutting fascia. Furthermore, any disclosure and/or feature(s) afforded to the/a circular cutting arrangement in the present application, according to this aspect, may be afforded to the/a cutting arrangement, whether it is circular or not, (eg whether it comprises a plurality of parts, and/or is movable/openable, etc).

According to a further aspect, there is provided an instrument for use in laparoscopic surgery, comprising: a cutting arrangement for making a cut to fascia; wherein the cutting arrangement is partially or wholly hollow. The cutting arrangement may, or may not, comprise gaps. According to this aspect, the cutting arrangement may, or may not, be circular. All other feature(s) and/or combination of feature(s) disclosed in the present application (eg spreading of the cut) are optional with regards to this broad aspect/statement of invention. Furthermore, any disclosure and/or feature(s) afforded to the/a circular cutting arrangement in the present application, according to this aspect, may be afforded to the/a cutting arrangement, whether it is circular or not, (eg whether it comprises a plurality of parts, and/or is movable/openable, etc). This statement of invention should in no way affect searching and examining of any more narrow appended claims.

According to a further aspect, there is provided an instrument for use in laparoscopic surgery, comprising: a cutting arrangement for making a cut to fascia. According to this aspect, the cutting arrangement may, or may not, be circularly rotatable, to facilitate cutting through fascia. Preferably the cut made to fascia is circular. The cutting arrangement may, or may not, be a circular cutting arrangement. The cutting arrangement may, or may not, be for circular cutting. The cutting arrangement may, or may not, be circular. The cutting arrangement may, or may not, comprise gaps. All other feature(s) and/or combination of feature(s) disclosed in the present application (eg spreading of the cut) are optional with regards to this broad aspect/statement of invention. Furthermore, any disclosure and/or feature(s) afforded to the/a circular cutting arrangement in the present application, according to this aspect, may be afforded to the cutting arrangement, whether it is circular or not, (eg whether it comprises a plurality of parts, and/or is movable/openable, etc). This statement of invention should in no way affect searching and examining of any more narrow appended claims.

According to a further aspect, there is provided an instrument for use in surgery, comprising: a cutting arrangement; and an arrangement to facilitate spreading a cut made by the cutting arrangement. According to this aspect, the cutting arrangement may, or may not, be circular. The cutting arrangement may, or may not, be rotatable, to make a cut. (It may make a cut in any way, with any type of cutting arrangement). According to this aspect, the instrument may, or may not, be for use in laparoscopic surgery. (It may be used in any type of surgery). According to this aspect, the instrument may, or may not, be used to cut through fascia and/or skin. Thus it may, or may not, be used for entry incisions/cutting, to gain entry into a patient's body. It will be known that, in surgery, after a cut is made, specialist other instrument(s) tend to be used to spread a cut. Thus it may be extremely beneficial to provide an instrument that carries out both cutting, and spreading of a cut. Any arrangement to facilitate spreading a cut made by the cutting arrangement may be provided, in no way limited to the example(s) shown. All other feature(s) and/or combination of feature(s) disclosed in the present application are optional with regards to this broad aspect/statement of invention. Furthermore, any disclosure and/or feature(s) afforded to the/a circular cutting arrangement in the present application, according to this aspect, may be afforded to the cutting arrangement, whether it is circular or not, (eg whether it comprises a plurality of parts, and/or is movable/openable, etc). This statement of invention should in no way affect searching and examining of any more narrow appended claims.

### Enlarged Summary

An example embodiment of the instrument relates generally to a surgical system. An example embodiment of the invention could be a system comprising of a surgical instrument that could be utilized to achieve a novel method of abdominal entry for laparoscopic surgery possibly allowing for resistance free penetration of the abdominal wall and negligible risk of inadvertent injury to intra-abdominal viscera (as a result of penetration of the abdomen). An example embodiment would not be intended to replace existing technology, but may potentially serve as a simple/cost-efficient, safe, and rapid method of direct abdominal entry (the first step of laparoscopic surgery). An example embodiment may comprise a hollow stainless steel tube with a 2 mm diameter, onto the tip of a standard curved hemostat, although other handle configurations may also be possible. Consequently, the tip of the hemostat could be a hollow tube that could be further modified by creating a ‘circular razor’ at its hollow tip. Another possible feature of the hollow tip is that it could separate into two semi-circular configurations when the hemostat is opened to allow the surgeon to ergonomically penetrate the abdominal fascia, perpendicular to the plane of the fascia, while maintaining optimal manual and sensory control of the example embodiment. The integrity of the abdominal wall is anatomically maintained by the fascia; the fascia is the layer of resistance which must be penetrated in order to achieve abdominal entry. The two ‘fingertips’ would be customized to serve as the side walls of the hollow shaft of the 2 mm stainless steel tube with a circular razor at its tip; this example incorporates the stated arrangement onto the tip of the curved hemostat. ‘One click’ of the hemostat handle achieves a locked position resulting in the creation of a circular hollow cutter at the tip. Manual unlocking of the hemostat (after fascial penetration) would allow the circular blade to separate into half circles, as fascia is bluntly opened to desired size, allowing for possible resistance-free introduction of a laparoscopic port. A possible embodiment consists of several possible components as described. A stainless steel mold may be constructed to duplicate the configuration of the customized tip. The entire surgical instrument could be made from surgical/disposable stainless steel, allowing for sterilization using standard operating room methods worldwide. A possible embodiment may work as follows: a small skin incision is made on the abdominal wall. The surgeon introduces the customized tip of the curved hemostat into the skin incision until resistance is encountered. The resistance is indicative of contact with the abdominal fascia. A twisting/circular turn of the tip of the hemostat along with simultaneous manual pressure applied along the vertical axis of the instrument may be implemented by the surgeon to penetrate the fascia (with the hollow circular razor tip). As soon as the fascia has been completely penetrated, the surgeon will easily sense the loss of resistance to forward penetration; notable is that the ‘circular razor’ at the tip of the hemostat offers minimal contact of instrument to tissue, thus negligible risk of injury to underlying tissue. Opening the hemostat (separating the hollow shaft of the punch into two half circles), bluntly enlarges the opening of the abdominal fascia to desired size. The blunt tipped laparoscopic working port is then inserted into the abdomen with no resistance, minimizing risk for injury to underlying viscera/vascular structures. Although the invention has been explained in relation to an example embodiment, it is to be understood that many other possible modifications and variations can be made without departing from the spirit and scope of the invention.

1. An instrument for use in laparoscopic surgery, comprising:
a circular cutting arrangement for making a circular cut to fascia;
wherein the circular cutting arrangement is circularly rotatable, to facilitate cutting through fascia.

2. An instrument for use in laparoscopic surgery as claimed in claim 1, wherein a portion or a whole of the instrument is circularly rotatable to circularly rotate the circular cutting arrangement.

3. An instrument for use in laparoscopic surgery as claimed in claim 1, wherein the circular cutting arrangement comprises a plurality of cut parts.

4. An instrument for use in laparoscopic surgery as claimed in claim 3, wherein the circular cutting arrangement comprises two substantially semi-circular cut parts.

5. An instrument for use in laparoscopic surgery as claimed in claim 3, wherein at least one or all of the plurality of cut parts are movable, to spread a cut made by the circular cutting arrangement.

6. An instrument for use in laparoscopic surgery as claimed in claim 5, wherein the instrument comprises an arrangement to facilitate moving the at least one or all of the plurality of cut parts of the circular cutting arrangement.

7. An instrument for use in laparoscopic surgery as claimed in claim 6, wherein the arrangement to facilitate moving the at least one or all of the plurality of cut parts comprises an opposing arrangement.

8. An instrument for use in laparoscopic surgery as claimed in claim 7, wherein the opposing arrangement comprises a scissor arrangement.

9. An instrument for use in laparoscopic surgery as claimed in claim 8, wherein the scissor arrangement comprises opposing handle parts.

10. An instrument for use in laparoscopic surgery as claimed in claim 9, wherein each opposing handle part comprises a finger holder.

11. An instrument for use in laparoscopic surgery as claimed in claim 1, wherein the circular cutting arrangement is movable from a closed position, to an open position.

12. An instrument for use in laparoscopic surgery as claimed in claim 1, wherein the instrument comprises a cut-spreading arrangement, for spreading a cut made by the circular cutting arrangement.

13. An instrument for use in laparoscopic surgery as claimed in claim 12, wherein the circular cutting arrangement comprises a plurality of cut parts, and the cut-spreading arrangement is configured to facilitate opening the plurality of cut parts of the circular cutting arrangement.

14. An instrument for use in laparoscopic surgery as claimed in claim 1, wherein the circular cutting arrangement is partially or wholly hollow.

15. An instrument for use in laparoscopic surgery as claimed in claim 1, wherein the instrument comprises a locking arrangement to lock the circular cutting arrangement in a closed position.

16. An instrument for use in laparoscopic surgery as claimed in claim 15, wherein the locking arrangement comprises an interlocking arrangement.

17. An instrument for use in laparoscopic surgery as claimed in claim 16, wherein the interlocking arrangement comprises at least one tooth.

18. An instrument for use in laparoscopic surgery as claimed in claim 1, wherein a directional axis of the circular cutting arrangement is different from a portion, or most, of the instrument.

19. An instrument for use in laparoscopic surgery as claimed in claim 1, wherein a portion or a whole of a half of the instrument deviates in angle from a portion of, or most of, the instrument, the half being the half that includes the circular cutting arrangement.

20. An instrument for use in laparoscopic surgery as claimed in claim 1, wherein a portion or a whole of a half of the instrument is curved, the half being the half that includes the circular cutting arrangement.
